(19) United States
(12) Patent Application Publication
(10) Pub. No.: US 2025/0127692 A1
(43) Pub. Date: Apr. 24, 2025

(54) Liquid Makeup Compositions

(71) Applicant: ELC Management LLC, Melville, NY (US)

(72) Inventors: John Castro, Huntington Station, NY (US); William Gately, Montclair, NJ (US); Carolyn Wang, Brooklyn, NY (US)

(21) Appl. No.: 18/492,328

(22) Filed: Oct. 23, 2023

(51) Int. Cl. A61K 8/06; A61K 8/25; A61Q 1/02

(52) U.S. Cl. CPC A61K 8/064; A61K 8/25; A61Q 1/02; A61K 2800/30; A61K 2800/43; A61K 2800/524

(57) ABSTRACT

Cosmetic compositions are water-in-silicone emulsions that are free of D4 and D5. The external phase comprises a solvent system of methyltrimethicone and low viscosity dimethicone; a film former system comprising trimethylsiloxysilicate (TMS); rheology modifiers; a pigment system that comprises one or more pigments that are surface treated with triethoxycaprylylsilane; and a first part of a sebum-controlling system that comprises spherical and ellipsoidal silica shells. The aqueous phase may comprise a second part of the sebum-controlling system, water-soluble skin care actives and a preservative system.

### FIELD

The present invention is in the field of topical skin compositions, specifically color cosmetics, such as liquid foundations and concealers, that exhibit stable color and transfer resistance over time.

### BACKGROUND

Formulation of commercially viable color cosmetic compositions is made challenging by various and sometimes competing requirements. These include color stability, transfer-proofness, sufficient play time, maintaining a matte finish, comfort, regulatory compliance, and overall product stability. We may consider two types of color stability. The first is shelf-life; how well or poorly a composition maintains its color in the container, weeks or months before it is applied to a skin surface. Another threat to product stability occurs as a result of contact with the skin. In the hours following application, skin sebum, perspiration and tears have deleterious effect on both color and finish (matte versus shiny), generally causing a shift in the color of the applied composition, and an overly shiny appearance. Furthermore, applied makeup may be destabilized by skin secretions that cause the makeup to run.

For some time now, the cyclomethicones, octamethylcyclotetrasiloxane (known simply as D₄) and decamethylcyclopentasiloxane (known as D₅), have been used as solvents in water-in-silicone makeup compositions. D4 is the more volatile of the two. At room temperature, D₄ dries, and forms a film on the skin that renders makeup coverage more stable and durable, conferring transfer-proofness to the makeup. In contrast, D₅ is only weakly volatile at room temperature, but offers certain benefits including as an emollient, humectant and viscosity controlling agent. However, D₄ and D₅ have been under regulatory scrutiny for their suspected persistence in the environment. This has led to a desire to remove D₄ and D₅ solvents from commercial cosmetics.

In making pigmented water-in-silicone emulsion compositions, the pigments are usually ground and predispersed in a silicone solvent. This preliminary step generally improves color strength by ensuring optimal dispersion of the pigment in the final composition. To facilitate wetting of the pigments by the solvent, the pigments may be surface treated. Dispersants may also be used to prevent agglomeration of previously dispersed pigment. The sizes to which pigment particles should be milled before being incorporated into a cosmetic composition is a function of the chemical identity of the pigment itself, as well as the specific surface treatment, dispersant and solvent being used.

Finding a way to achieve color stability, transfer-proofness, sufficient play time and opacity while maintaining a matte finish, comfort, and overall product stability without the use of D₄ and D₅ has not been a straightforward endeavor for the industry. There remains, therefore, a need for commercially viable cosmetic water-in-silicone emulsion compositions that achieves all of these objective without the use of D₄ and D₅.

### OBJECT OF THE INVENTION

A main object of the invention is to provide commercially viable cosmetic makeup compositions that exhibit color-fastness, transfer resistance, opaqueness and a pleasant feel over time, while being resistant to the degrading effects skin secretions, and without the use of D₄ and D₅.

### SUMMARY

Color cosmetic compositions according to the present invention comprise a solvent system of methyltrimethicone and low viscosity dimethicone; a film former system comprising trimethylsiloxysilicate (TMS); a pigment system that comprises one or more pigments that are surface treated with triethoxycaprylylsilane; and a sebum-controlling system comprising one or more of silica shells, platy mica, and sebum regulators. The compositions are water-in-silicone emulsions, and may also comprise various skin benefit agents, but no D₄ or D₅.

### DETAILED DESCRIPTION

Throughout the specification, the term “comprises” and its cognates means that a set of elements may not be limited to those elements explicitly recited. All numerical concentrations and ratios are modified by the term “about” unless explicitly stated otherwise. All percentages, parts and ratios are based upon the total weight of the compositions of the present invention, unless otherwise specified. The term “weight percent” may be denoted as “wt. %” herein.

As used herein, the term, “volatile solvent” means a solvent that evaporates quickly at room temperature, preferably, at a rate of about 0.01 mg/cm²/min or more. The term, “nonvolatile solvent”, as used herein means a solvent which does not quickly evaporate at room temperature. Preferably, a nonvolatile solvent will have an evaporation rate of about 0.002 mg/cm²/min or less. The evaporation rates herein are expressed in mg of solvent evaporated per unit area (cm²) per unit time (minute).

The term, “water-soluble”, as used herein, means that a polymer is soluble in water. In general, a polymer should be soluble at 25° C., at a concentration of at least 0.1 wt. % of the water solvent, preferably at least 1 wt. %, more preferably at least 5 wt. %, most preferably at least 15 wt. %. The term “water-insoluble,” as used herein, means that a compound is not soluble in water. Thus, the compound is not miscible with water.

### External Phase

Makeup compositions of the invention comprise an external, silicone-based phase. The external phase typically comprises about 50% to about 75% by weight of the of the total makeup composition. The external phase comprises a solvent system, a film former system, rheological modifiers, pigments, a first part of a sebum-controlling system, and optionally, agents that benefit the skin or the overall performance of the composition.

### Solvent System

The solvent system is crucial to pigmented color cosmetics. In the first instance, the solvent wets the color pigments for easier and uniform incorporation into the composition. Generally, however, as the solvent system dries, the cosmetic composition undergoes color shift. This shift in color needs to be optimized to so that a user can be sure of the end result of makeup application. For example, in reformulating an existing commercial product for D4 and D5 removal, the goal may be to create a new composition in which the color shift is similar to the color shift of the existing product. Also, the solvent system should evaporate at a rate that allows enough “play” time for a user to apply and spread the composition over her skin, while not requiring so much time so that the user feels wet for an extended period.

We have found that a solvent system that comprises certain combinations of methyltrimethicone and low viscosity dimethicone are very beneficial as a solvent system without cyclomethicones. Methyl trimethicone (C₁₀H₃₀O₃Si₄) is a volatile, branched silicone fluid, having a viscosity of about 1.5 centi-Stoke (cSt) at room temperature. Dimethicone is a linear silicone fluid which viscosity and volatility depend on polymer chain length. In general, dimethicone viscosities ranges from one or less to millions of cSt. In the present invention, dimethicone viscosity should not exceed 5 cSt, but particularly preferred is a viscosity between about 1 cSt and about 2 cSt. With these restrictions, useful results may be achieved when the ratio of methyltrimethicone to dimethicone is between about 3:2 and 4:1, preferably between 3:2 and 2:1. A most preferred ratio of methyltrimethicone to dimethicone for achieving the goals of the present invention is between 3:2 and 7:4. The combined concentration of methyltrimethicone and dimethicone will range from about 10% to about 40% by weight of the total makeup composition; for example, a concentration of about 15% to about 30% by weight of the total makeup composition. More preferred is a concentration of about 20% to about 25% by weight of the total makeup composition This well-defined solvent system, in combination with the film former system and treated pigments described below, delivers excellent wetting with stable shade upon drying, while providing a reasonable amount of play time for application and high opacity for covering skin imperfections.

### Film Former System

The film former subsystem of the present invention comprises trimethylsiloxysilicate (TMS). TMS (C₆H₁₈O₅Si₃) is a transparent crosslinked MQ silicone resin polymer known to be used in cosmetic products. In the present invention, powdered or flaked trimethylsiloxysilicate is dispersed in the methyltrimethicone/dimethicone solvent system described above. When this dispersion of TMS is spread on a surface and allowed to dry, it forms a film that has a high substantivity and non-tacky feel. The film is effective to hold suspended pigments in place, and confer transfer resistance. It was not immediately clear that there existed a ratio of methyltrimethicone to dimethicone (as given above), that when combined with some concentration of TMS resin would give a beneficial result, in terms of color stability, dry-time, transfer resistance, product feel and overall product stability. Nevertheless, we have found useful concentrations of TMS resin in a makeup compositions according to the present invention. For example, with the concentrations and ratios of methyltrimethicone and low viscosity dimethicone given above, useful concentrations of TMS may range from about 2% to about 15% by weight of the total makeup composition. Preferred is a concentration of about 5% to about 12%, for example, about 8% to about 10% by weight of the total makeup composition.

### Contrast Ratio as a Function of TMS Concentration

Makeup compositions according to the present invention, each with a different amount (3%, 5%, 10%) of TMS, were prepared. For each composition, two thin-film samples of 1 mil (0.0254 mm) thickness were prepared by spreading the composition on the black and white areas of a contrast ratio chart. The samples were allowed to dry for 24 hours. Thereafter, the CIE Y tristimulus value of each sample was measured. The Y value represents luminance, and the ratio of Y values, Y_{black}/Y_{white}, gives the contrast ratio, wherein a higher contrast ratio corresponds to a more opaque material. It was noted that as the concentration of TMS increased, the opacity of the makeup composition also increased, which enhances makeup coverage and the ability to hide skin imperfections. This is beneficial, but wholly unexpected because TMS is transparent, and could not be expected to have any effect on opacity or on the ability of the makeup composition to cover skin imperfections. In fact, in the test samples just described, the increase in opacity of the film with increasing TMS is easily visible to the naked eye. That transparent TMS could be used to improve the ability of the makeup compositions to hide skin imperfections was unexpected. Also, when reformulating a commercially available composition to remove D4 and D5, the goal may be for the reformulated product to mimic the behavior of the original product. The ability to meet this goal is made easier by adjusting the level of TMS to achieve an opacity that is similar to the original product. But again, this was unexpected, especially because TMS is transparent.

### Rheological Modifiers/Suspending Agents

Compositions according to the present invention will typically comprise one or more rheological modifiers and/or suspending agents. Organoclays, such as disteardimonium hectorite and stearalkonium bentonite are known to be useful. Rheological modifiers and/or suspending agents will typically be used up to about 3% by weight of the total makeup composition. Hectorites are typically processed with the addition of polar activator that aids the dispersion of the organoclay and enhances stability. Examples of useful polar activators include triethyl citrate, propylene carbonate, propylene carbonate/water (95/5), and ethanol/water (95/5). The concentration of polar activator will typically by about 30% to about 50% of the concentration of organoclay.

### Pigments

The color, shade of the final composition is achieved with one or more cosmetic grade powdery pigments that are dispersed in the composition. The ability to achieve consistent, stable color depends, in part, on achieving good dispersion of the pigment throughout the composition. With the solvent and film former systems described above, we have found that pigments that have been surface-treated with triethoxycaprylylsilane provide very stable true color. By “true color” we mean that there is predictable color shift upon drying. This predictability is particularly beneficial when reformulating for D4 and D5 removal, where the goal is for the reformulated product to match the color shift behavior of the original product. In tests, pigments that were surface treated with triethoxycaprylylsilane performed better than pigments treated with methicone, for example. Useful pigments are inorganic and insoluble in the solvent system in which they are dispersed. Examples of useful pigments include iron oxides and titanium dioxide and mica. In makeup compositions according to the present invention, the total amount of all pigments coated with triethoxycaprylylsilane may range from about 5% to about 20% by weight of the total makeup composition.

### Sebum-Controlling System

As noted above, one of the challenges of making a color-true, stable makeup composition is skin contact. In the hours following application, skin sebum, perspiration and tears have deleterious effect on both color and finish (matte versus shiny), generally causing a shift in the color of the applied composition, and an overly shiny appearance. Furthermore, applied makeup may be destabilized by skin secretions that cause the makeup to run. Therefore, we have developed an effective sebum-controlling system that does not interfere with the realized benefits of the makeup compositions as thus far described.

A first part of a sebum-controlling system is a sebum-absorbing system that is added to the external phase, and comprises hollow spherical and hollow ellipsoidal shells formed of porous silica, that meet well-defined specifications. Preferred are spherical shells have an average size of 10-14 μm and an oil absorbing capacity of at least about 100 grams of oil per 100 grams of spherical silica shells. Preferred ellipsoidal shells have an average size of about 2-4 μm and an oil absorbing capacity of at least about 450 grams of oil per 100 grams of ellipsoidal silica shells. We have found that this combination of silica shells in certain proportions give excellent results. Silica particles that meet these specifications area available from Kobo Products, for example. It was not obvious that an effective sebum-controlling system could be developed that would not destabilize the color, stability and sensorial experience of the makeup compositions described herein. For maximum effectiveness, the ratio of spherical silica shells to ellipsoidal silica shells (of the types described above) should be between about 1:1 and 4:1, preferably about 2:1 to 3:1. This relatively narrow range of ratio is effective to absorb sebum and prevent it from destabilizing the applied makeup composition, thus maintaining a powdery, matte appearance without color shifting. Generally, the concentration of spherical and ellipsoidal silica shells, of the types described above, will each range from about 0.1% to about 2.0% by weight of the total makeup composition. Optionally, the sebum-absorbing system may also comprise mica platelets up to about 1% by weight of the of the total makeup composition.

### Skin-Benefit Agents

Optionally, the internal phase may comprise agents that benefit the skin or the overall performance of the composition. These include, but are not limited to, moisturizers, conditioning agents, lubricants, agents that improve the texture of the skin or the composition, skin barrier agents, sun screen agents and skin repair agents.

### Internal Phase

Compositions of the invention comprise an internal aqueous phase. The aqueous phase may comprise a second part of the sebum-controlling system, water-soluble skin care actives and a preservative system. The aqueous phase comprises water, typically from about 25% to about 75% of the total makeup composition.

As noted above, the present invention comprises a first part of a sebum-controlling system that is added to the external phase. Optionally, but preferably, compositions according to the present invention also comprises a second part of a sebum-controlling system that is added to the aqueous phase. This second part may comprise one or more sebum regulators that are effective to reduce or prevent the secretion of sebum onto the skin surface. Sebum regulators, which may be present in the internal phase up to about 5% of the total makeup composition, include, for example, Laminaria saccharina up to about 1%, and niacinamide up to about 3% by weight of the of the total makeup composition.

Other ingredients that may be present in the aqueous phase include water-soluble skin care actives that provide a benefit to the skin or to the overall performance of the makeup composition. Many such actives are known in the art, but we especially mention resveratrol, hyaluronic acid and Lactobacillus ferment, each up to about 2% by weight of the of the total makeup composition. Typically, the aqueous phase will also comprise a preservative system up to about 2% by weight of the of the total makeup composition.

### Form of Composition

Liquid makeup compositions according to the present invention are in the form of water-in-silicone emulsions, and will comprise a surfactant system from about 2% to about 10% by weight of the of the total makeup composition. The surfactant system is effective to create water-in-silicone emulsions. Useful examples include lauryl PEG-9 polydimethylsiloxyethyl dimethicone and dimethicone/PEG-10/15 crosspolymer and PEG-10 dimethicone.

### Non-Limiting Example

The following is an example of a water-in-silicone liquid makeup composition. This example is not intended to be limit the present invention. Modifications can be undertaken by the skilled artisan without departing from the spirit and scope of this invention.

| | Percent |
|---|---|
| External Phase | |
| methyltrimethicone (1.5 cSt) | 15.64 |
| dimethicone (2 cSt) | 9.56 |
| trimethylsiloxysilicate | 10.00 |
| disteardimonium hectorite | 0.60 |
| triethyl citrate | 0.20 |
| spherical silica shells (12 μm, 119 gm/100 gm)¹ | 0.25 |
| ellipsoidal silica shells (3 μm, 490 gm/100 gm)² | 0.25 |
| pigments coated with triethoxycaprylylsilane | 13.00 |
| Emulsifiers | |
| PEG-10 dimethicone | 2.20 |
| lauryl PEG-9 polydimethylsiloxyethyl | 2.00 |
| dimehicone | |
| dimethicone/dimethicone\PEG-10/15 | 2.00 |
| crosspolymer | |
| Internal Phase | |
| water | 32.60 |
| niacinamide | 2.50 |
| laminaria saccharina extract | 0.20 |
| water soluble skin care actives | 8.00 |
| Germazide PSG³ | 1.00 |

¹MSS-500W from Kobo Products, Inc.
²Shells-SH from Kobo Products, Inc. (with methoxy amodimethicone/Silsesquioxane copolymer coating)
³phenoxyethanol / chlorphenesin / glycerin / sorbic acid

Compositions according to the above formula satisfy the main objectives of the present invention, specifically to provide commercially viable cosmetic makeup compositions that exhibit color-fastness, transfer resistance, and a pleasant feel over time, while being resistant to the degrading effects skin secretions, and without the use of D₄ and D₅. As an unexpected benefit, the opacity (ability of the finished makeup product to hide skin imperfections) can be fine tuned by adjusting the concentration of TMS.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A water-in-silicon emulsion makeup composition that comprises an external phase and an internal aqueous phase, the external phase comprising:
a solvent system comprising methyltrimethicone and low viscosity dimethicone;
a film former system comprising trimethylsiloxysilicate;
one or more rheology modifiers;
one or more pigments that are surface treated with triethoxycaprylylsilane; and
a first part of a sebum-controlling system that comprises spherical silica shells and ellipsoidal silica shells;
wherein the composition comprises no octamethylcyclotetrasiloxane (D₄) or decamethylcyclopentasiloxane (D₅).

2. The water-in-silicon emulsion makeup composition of claim 1 wherein the viscosity of the low viscosity dimethicone is between 1 and 2 cSt, and the ratio of methyltrimethicone to low viscosity dimethicone is between 3:2 and 4:1.

3. The water-in-silicon emulsion makeup composition of claim 2 wherein the combined concentration of methyltrimethicone and low viscosity dimethicone will range from 10% to 40% by weight of the makeup composition.

4. The water-in-silicon emulsion makeup composition of claim 3 wherein the concentration of trimethylsiloxysilicate ranges from 2% to 15% by weight of the total makeup composition.

5. The water-in-silicon emulsion makeup composition of claim 3 wherein the total concentration of all pigments coated with triethoxycaprylylsilane is between 5% and 20% by weight of the total makeup composition.

6. The water-in-silicon emulsion makeup composition of claim 1 wherein the spherical silica shells have an average size of 10-14 μm and an oil absorbing capacity of at least 100 grams of oil per 100 grams of spherical silica shells, and the ellipsoidal silica shells have an average size of 2-4 μm and an oil absorbing capacity of at least 450 grams of oil per 100 grams of ellipsoidal silica shells.

7. The water-in-silicon emulsion makeup composition of claim 6 wherein:
the concentration of spherical silica shells and ellipsoidal silica shells is between 0.1% and 2.0% by weight of the total makeup composition; and
the ratio of spherical silica shells to ellipsoidal silica shells is between 1:1 and 4:1.

8. The water-in-silicon emulsion makeup composition of claim 2 wherein the aqueous phase comprises:
water from 25% to 50% of the total makeup composition;
a second part of the sebum-controlling system that comprises one or more sebum-controlling agents;
water-soluble skin care actives, and
a preservative system.

9. The water-in-silicon emulsion makeup composition of claim 8 wherein the one or more sebum controlling agents is Laminaria saccharina up to about 1%, and niacinamide up to about 3% by weight of the of the total makeup composition.
